(19) United States
(12) Patent Application Publication
(10) Pub. No.: US 2002/0131988 A1
(43) Pub. Date: Sep. 19, 2002

(54) Pharmaceutical implant containing immediate-release and sustained-release components and method of administration

(76) Inventors: Todd P. Foster, Kalamazoo, MI (US); William M. Moseley, Augusta, MI (US); Satish K. Singh, Portage, MI (US)

Correspondence Address: Andrew M Solomon, Pharmacia & Upjohn Company, Global Intellectual Property, 301 Henrietta Street, Kalamazoo, MI, 49001, US

(21) Appl. No.: 09/500,246

(22) Filed: Feb. 8, 2000

Related U.S. Application Data
(60) Provisional application No. 60/171,215, filed on Dec. 16, 1999.

(51) Int. Cl. A61F 13/00; A61F 2/00; A61K 9/00

(52) U.S. Cl. 424/422; 424/425; 424/426; 424/427; 424/428; 424/429

(57) ABSTRACT

A pharmaceutical implant for administering a biologically active substance is made up of an immediate-release component, preferably containing a disintegrating agent, and a sustained-release component. The implant of the present invention provides flexibility in adjusting the release of the medicament and a faster onset of release can be provided along with a long-term sustained-release. The release rate of the biologically active substance can be adjusted by controlling the relative quantities of the immediate-release component and the sustained-release component.

### CROSS-REFERENCE TO RELATED APPLICATIONS

This application claims the benefit of the following provisional application: U.S. Ser. No. 60/171,215, filed Dec. 16, 1999, under 35 USC 119(e)(i).

### BACKGROUND OF THE INVENTION

1. Field of the Invention

This invention relates to a pharmaceutical implant composition and a method of administering a biologically active substance using this implant composition and, more specifically, to a pharmaceutical implant composition comprising an immediate-release component and a sustained-release component wherein the components are maintained as discrete, separate physical entities.

2. Technology Description

The implantation of a biologically active substance has long been favored as a method of obtaining a sustained release of the biologically active substance into the system of a subject to be treated where a long duration of action is required and where the normal oral route may not be sufficiently effective, would require frequent administration, or may be associated with gastric side-effects.

A substantial body of literature exists on sustained or controlled release dosage forms suitable for administration as an implant. Therapeutic classes where implants are particularly well suited, include among others, contraceptive steroids, peptide hormones, prostaglandins, narcotic antagonists, anti-arrhythmics, and anti-cancer agents. Ballard and Nelson in J. Pharm. Sci., 51, 915-924 (1962) discuss the theories for absorption of implanted solid drug. Gangadharam et al. in J. Controlled Release, 26, 87-98 (1993) disclose an implant made of a biodegradable polymer for the sustained release of an anti-mycobacterial drug. Yamanaka et al. in J. Pharm. Biomed. Anal. 15, 1851-1859 (1997) show the advantages of a subcutaneous delivery of an angiotensin-converting enzyme inhibitor Imidaprilat via an implanted osmotic pump. A safe and effective treatment for endometriosis is the gonadotropin-releasing hormone agonist delivered via a subcutaneous implant formed of biodegradable polymers based on poly(lactic-co-glycolic)acid.

In animals, hormonal implants are used to enhance growth and improve carcass quality. U.S. Pat. No. 3,417,182 discloses the implanting of pellets of melengestrol acetate, hereinafter referred to as MGA, into cattle to increase the weight of the cattle. Henricks et al in the Journal of Animal Science, (1997), 75, 2627-33, discloses the implantation of trenbolone acetate (TBA) and the feeding of melengestrol acetate to heifers to increase the weight gain thereof. French Patent 2 290 906 discloses a hormone composition containing estrogen and progesterone which accelerates the growth and fattening of animals. U.S. Pat. No. 3,737,521 discloses the use of a solid cylindrical rod having a linear polyetherurethane matrix containing an estrus-blocking progestational hormone which is implanted in the neck-tissue of fertile heifers to control the onset of estrus and ovulation. U.S. Pat. No. 4,708,874 discloses a device that can be implanted for the controlled release of drugs or nutrients. Jones et al. in J. Controlled Rel., 30, 35-44 (1994) discuss the efficacy of a biodegradable-polymer based metoclopramide implant to prevent fescue toxicosis in cattle. Shih et al., in J. Controlled Rel., 25, 155-162 (1993) implanted ivermectin in dogs in bioerodible poly(orthoester) matrices. Doasy et al., Int. J. Pharm., 89, 251-259 (1993) designed and evaluated a biodegradable poly(lactic-co-glycolic)acid copolymer based implant for the delivery of estradiol to steers. U.S. Pat. No. 5,744,163 discloses a sustained-release implant formulation of an animal growth hormone based on a tablet coated with a biodegradable polymer and a poloxamer.

Release of drugs from pellet or tablet based implants is driven primarily by the solubility of the drug in the plasma or fluids at the implantation site and the effective surface area of the dosage form. The rate is determined by the solubility and effective surface area while the duration of release is a function of the amount of drug load in the pellets. The initial drug release rate is not specifically controlled to any extent, but simply becomes a function of the formulation that is designed primarily from the point of view of providing a long-term release. The initial release rate is not a design criterion. U.S. Pat. No. 5,874,098 teaches a multi-pellet implant for administering a sustained release pharmaceutical active and an antibiotic for treating the injection site. The multiple pellets must contain different active materials.

Release from other implants based on a rate-limiting matrix, e.g., cholesterol or silastic elastomer, is determined by the rate of diffusion in the matrix forming material. Examples of these are well represented in the literature, e.g., Opdebeeck and Tucker, Int. J. Pharm., 23, 271-279 (1993). These implants tend to have a burst-phase arising simply because a small part of the drug happens to be immobilized at the surface of the matrix during the fabrication. The burst-phase is often considered an undesirable phenomena to be minimized before the pseudo steady-state phase is achieved. A polymer coating is often used to overcome this burst-effect.

Release from implants based on biodegradable polymers such as poly(lactic-co-glycolic)acid is based primarily on the rate of degradation of the polymer. Again, a burst-effect is often seen resulting from the part of the drug in close proximity to or on the surface, which is a function of the manufacturing process and to some extent the composition of the implant.

U.S. Pat. No. 2,895,875 discloses a preparation that exerts a strong initial and subsequently a prolonged hormone activity for implantation in human and veterinary therapy. However, the method of providing for this is via a relatively complicated process of producing pellets with an inner core of coarse hormone crystals surrounded by a layer of smaller more rapidly dissolving crystals in a binder such as methylcellulose.

Despite the above described advances in the art, there is a need for a combination of rapid onset of action as well as the long-term delivery of the same biologically active agent in the form of an implant. While this may not be of concern in a number of situations involving long-term therapy, e.g., anti-cancer, there are others such as contraception or immunization where a rapidly delivered initial dose followed by a slower sustained dosing will provide a therapeutic advantage. For example, a rapidly delivered dose of a contraceptive may inhibit the occurrence of early unwanted pregnancies that may occur following administration of a sustained release contraceptive which requires a considerable period of time to reach therapeutically effective levels. Similarly, a burst delivery of a vaccine followed by slow delivery may obviate the need for external adjuvants to achieve significant levels of immune response.

In food animal implants, the need for a rapid onset of action often requires that a high dose be given in the implant. This is however associated with the risk of unacceptably high tissue and fat residues of the substance. The improved implant system of the present invention alleviates this drawback.

Accordingly, there exists a need in the art for an implant containing two distinct delivery vehicles for the same biologically active material, namely a first vehicle containing a “fast acting” or “immediate-release” form of the active material, and a second vehicle containing a sustained release version of the same active.

### BRIEF SUMMARY OF THE INVENTION

It is an object of the present invention to provide an improved pharmaceutical single injection implant containing separate delivery vehicles for the same biologically active material wherein a first vehicle is capable of providing a rapid release and thus a rapid onset of action of the active substance, and wherein the second vehicle is capable of providing a sustained release of the same substance.

It is a further object of the present invention to provide a pharmaceutical implant system that allows the total release rate from the implant to be modulated in a simple manner, thus also modulating the total duration of effectiveness of the implant.

It is another object of the present invention to allow the total dose administered to be reduced while still achieving a rapid onset of action.

It is a further object of the present invention to provide an effective implant system for food animals which provides an ability to control residue levels in tissues and fat while achieving pharmacological efficacy directly after implantation.

These and other objects of the present invention are met by providing a pharmaceutical single injection implant for and a method of administering a biologically active substance to the subject in which the same biologically active substance is provided in two separate delivery vehicles having differing release rates. In particularly preferred embodiments, the vehicles comprise one or more pellets containing a disintegrating agent and one or more pellets not containing a disintegrating agent.

### BRIEF DESCRIPTION OF THE DRAWINGS

FIG. 1 is a graph showing the release profile for the pellets of Example 1.

### DETAILED DESCRIPTION OF THE PREFERRED EMBODIMENT

In describing the preferred embodiment, certain terminology will be utilized for the sake of clarity. Such terminology is intended to encompass the recited embodiment, as well as all technical equivalents which operate in a similar manner for a similar purpose to achieve a similar result.

The present invention relates to an injection implant comprising two separate delivery vehicles of the same biologically active ingredient. The first vehicle is capable of providing an immediate-release of the ingredient to the animal system whereas the second vehicle is capable of providing a sustained or extended release of the same active.

By the term “implant” is meant any physical device containing the biologically active material in multiple delivery vehicles such that the vehicles are delivered to the animal's system via an injection. In most embodiments the implant contains the immediate-release and sustained-release vehicles such that they both be administered in a single injection, but embodiments where multiple injections of either the immediate-release and/or sustained-release vehicles occurring at different points in time is expressly covered.

The concept of injectable implants is well known to those skilled in the art and it is submitted that one could envision any of a number of embodiments designed to simultaneously deliver the multiple vehicles via a single injection. For example, an injectable implant system is described in U.S. Pat. No. 5,874,098. To the extent necessary for completion, this reference is expressly incorporated by reference.

The term “immediate-release” defines a vehicle that, within a finite period of time, for example 24 hours, releases in vivo enough of the biologically active material to begin to achieve a desired effect in the patient. For example, an implant which releases at least 30% percent of its active material within 24 hours as defined by the methodology of Example 1 could qualify as such a vehicle. The term “sustained-release” defines a vehicle that releases the same active material at a slower rate as compared to the “immediate-release” vehicle. For example, an implant which retains at least 30% percent of its active material within 24 hours as defined by the methodology of Example 1, provided that its release rate is slower than that of the immediate-release vehicle could qualify as such a vehicle. The concept of immediate-release and sustained-release compositions are known in the art. However, the use of an implant containing multiple delivery vehicles which can deliver the same active both immediately and over a sustained period of time is novel. Furthermore, the time period defined by “immediate-release” or “sustained-release” is often determined by the disease or disorder being treated. For example, for some diseases or disorders, an immediate-release will produce a desired effect in minutes or hours, whereas for other diseases or disorders, an immediate-release will produce a desired effect in a matter of days or weeks.

The first delivery vehicle comprises a delivery system capable of immediately releasing enough active material to generate a desired effect in a patient shortly after administration. There are many ways to design a vehicle capable of this and such vehicles are considered as being within the skill of the artisan. Examples of immediate-release vehicles include, but are not limited to the following: coated solids or liquids where the coating wall material is very thin, coated solids or liquids where the coating wall material is very soluble in body fluids, porous or freeze-dried solids having an increased surface area contact, a solid tablet or pellet containing a disintegrating agent which causes the solid tablet to rapidly break down when in body fluids, a solid or pellet containing a relatively small or micronized active particle size, an osmotic delivery system where the osmotic system is such that a substantial amount of the active is released upon implantation, and mixtures thereof. The above listing is considered merely representative and one skilled in the art could envision other immediate-release mechanisms/embodiments.

The second delivery vehicle comprises a sustained release delivery system. As a practical matter, the skilled artisan may select any of the following non-limiting sustained release delivery vehicles to contain the actives of the implant of the claimed invention: encapsulated solutions or suspensions, biodegradable solid substances, conventional tablet/pellet formulations optionally utilizing either disintegrating agents and/or active particle size to modulate release, conventional tablet/pellet formulations coated with a polymeric membrane to control release (e.g., ethylcellulose), matrix-tablets based on gel-forming excipients (e.g., hydroxypropyl methyl cellulose), matrix-type systems based on non-biodegradable polymers (e.g., medical grade silastics), membrane-type systems based on non-biodegradable polymers (e.g., medical grade silastics), matrix-type systems based on biodegradable polymers (e.g., polylactic acid and polyglycolic acid homo and copolymers of various compositions), matrix-type systems based on lipidic excipients (e.g., cholesterol, waxes), mass transfer systems based on osmotic pressure pumping through a hole in an impermeable coating and mixtures thereof. The above listing is considered merely representative and one skilled in the art could envision other sustained release mechanisms/embodiments.

In particularly preferred embodiments, the implant comprises a magazine containing solid biodegradable pellets containing the same actives and having differential release characteristics It is still further contemplated that a magazine containing greater than two pellets could be used in accordance with the present invention.

Selection of the specific implant embodiment is largely determined by the specific end result desired. In a preferred embodiment, the biologically active ingredient can be provided in the form of a immediate-release component containing a disintegrating agent and a sustained-release component that does not contain a disintegrating agent. The immediate-release component can be provided in the form of granules or pellets containing the biologically active ingredient and can be formed by conventional granulation practices or through direct compression processes. The pellets typically contain from about 1 to 99 wt. % of the biologically active ingredient with the remainder being conventional tableting ingredients such as magnesium stearate, stearic acid, colloidal silicon dioxide, talc, titanium dioxide, magnesium, calcium and aluminum salts, lactose, povidone, high molecular weight polyethylene glycols and derivatives thereof, bioerodible polymers such as poly(orthoesters) and polyanhydrides and anhydride co-polymers, polyoxystearates, carboxymethylcellulose, cellulose esters such as acetate phthalate, acetate succinate and cellulose acetate, N,N-diethylamino acetate, polyvinyl alcohol, hydroxypropyl methyl cellulose, and the like.

In the immediate-release vehicle, a disintegrating agent is also preferably present in order to enable the immediate-release of the pharmacologically active ingredient once it is implanted into the subject. Conventional disintegrating agents used in tableting processes can be used in the present invention with sodium crosscaramellose, sodium carboxymethylcellulose, microcrystalline cellulose, powdered cellulose, colloidal silicon dioxide, crospovidone, guar gum, magnesium aluminum silicate, methyl cellulose, alginic acid, calcium carboxymethylcellulose, potassium polacrilin (and other cation exchange resins such as Amberlite resins), starch, pregelatinized starch, sodium starch glycolate, and sodium alginate being especially preferred. The disintegrating agent typically is contained in the pellet in an amount of 0.1-50% by weight, based on the total weight of the pellet, with 0.5-15% by weight being preferred and 1-6% by weight being especially preferred.

The pellets are formed according to conventional methods that involve the mixing of the ingredients, wet, dry, or fluid-bed granulation, or extrusion/spheronization, followed by screening, drying, screening/sizing, lubrication and compression. These steps are well known in the art.

As discussed above, the implant dose is comprised of a combination of the two types of pellets. The time release properties of the implant composition can be controlled by varying the number of pellets containing the disintegrating agent with respect to the pellets not containing a disintegrating agent. The number of pellets containing a disintegrating agent and the number of pellets which do not contain a disintegrating agent in the implant composition can be readily determined depending on the drug being administered, the subject to whom the drug is being administered and the desired duration of treatment. Alternatively, differential active loadings can also be utilized to achieve desired results. The method of choice is considered as falling within the skill of the artisan.

In the present invention, the biologically active ingredient contained in the implant composition is not critical and can be any substance such as enzymes or other organic catalysts, ribozymes, organometalics, proteins and glycoproteins, peptides, poly(amino acids), antibodies, nucleic acids, steroids, antibiotics, antimycotics, anti-narcotics, cytostatics, cytotoxics, cytokines, carbohydrates, oleophobics, lipids, antihistamines, laxatives, vitamins, decongestants, gastrointestinal sedatives, anti-inflammatory substances, antimanics, anti-infectives, coronary vasodilators, peripheral vasodilators, cerebral vasodilators, psychotropics, stimulants, anti-diarrheal preparations, anti-anginal drugs, vasoconstrictors, anticoagulants, antithrombotic drugs, analgesics, antipyretics, hypnotics, sedatives, antiemetics, antinauseants, anticonvulsants, neuromuscular drugs, hyperglycemic and hypoglycemic agents, antivirals, antineoplastics antidepressants, anticholinergics, antiallergic agents, antidiabetic agents, antiarrythmics, antihormones, antihistamines, β-blockers, cardiac glycosides, contraceptives, contrast materials, radiopharmaceuticals, dopaminergic agents, lipid-regulating agents, uricoscurics, tranquilizers, thyroid and antithyroid preparations, diuretics, antispasmodics, uterine relaxants, mineral and nutritional additives, antiobesity drugs, hormones, antihelmentics, pharmaceuticals and other therapeutic agents. The invention may also be employed for the delivery of microorganisms, either living, attenuated or dead such as bacteria, and viruses such as indigenous vira, enterovira, bacteriophages. The present invention is especially suited for the immediate and sustained delivery of hormones and steroids such as androgens, such as testosterone, trenbolone acetate (TBA), dihydroepiandroterone, and other androgenic steroids, estrogens, such as estradiol-17-β, estradiol benzoate, zeralanone, and other estrogenic steroids, progestins, such as progesterone, melengestrol acetate (MGA), megestrol acetate, medroxyprogesterone acetate, norgestemet, norethidrone, and other progestin compounds, releasing factors, such as leutinizing hormone releasing hormone and analogs, growth hormone releasing hormone and analogs, thyroid releasing hormone and analogs, and other releasing factors and analogs, growth hormones/somatotropin, such as natural and recombinant somatotropins and analogs from various species, growth factors, such as insulin-like growth factor, epidermal growth factor and other such factors. It is also especially suited for delivery of antihelmintics, such as invermectins, and antigens. An especially preferred use of the present invention is in the suppression of estrus, inhibition of pregnancy and increased body weight of cattle through the implantation of the implant composition of the present invention in the body of the cattle containing MGA, a combination of MGA and TBA or a combination of MGA, TBA and estradiol as the biologically active ingredient. A preferred embodiment for this use comprises an implant containing one to four, more preferably one to two immediate-release pellets and four to six, more preferably three to five sustained-release pellets. An even more preferred embodiment for this use comprises an implant containing one immediate-release pellet and five sustained-release pellets.

In practice, the active ingredients are contained in the delivery vehicle, for example pellets, preferably in an amount of from 1 to 99% and preferably from 50 to 90 wt. %.

In particularly preferred embodiments, when used to administer MGA and/or TBA, the present invention can provide beneficial and advantageous results in the hormonal control of the reproductive cycle in animals, for example, by reducing the post-partum anestrual period in cattle; by synchronization of the estrual period in a group of cattle; by preventing estrual activity in fattening meat animals; by controlling the estrual period in individual animals; and by providing compositions and methods to further weight gain with lessened side effects in beef cattle. When MGA or TBA are the biologically active compositions, each delivery vehicle contains between about 5 to about 200 mg of MGA or TBA. In addition, the carcass composition of the animal may be improved; for example, a carcass having increased lean and less fat may result.

In addition to the active ingredients, each of the delivery vehicles of the implant may independently contain standard granulating aids such as lubricants, diluents, binders and glidants, magnesium stearate, stearic acid, colloidal silicon dioxide, talc, titanium dioxide, magnesium, calcium and aluminum salts, lactose, cyclodextrins and derivatives thereof, starches, povidone, high molecular weight polyethylene glycols and derivatives thereof, bioerodible polymers such as poly(orthoesters) and polyanhydride and anhydride co-polymers, polystearates, carboxymethyl cellulose, cellulose esters such as acetate phthalate, acetate succinate and cellulose acetate, N,N-diethylamine acetate, polyvinyl alcohol, hydroxypropyl methyl cellulose, other biologically active or inactive substances, other pharmaceutically active or inactive substances, and the like.

The implant composition of the present invention can be administered subcutaneously, intramuscularly, intraperitoneally, intracranially, etc., depending on the most desirable site of administration for the biologically active ingredient. In a particularly preferred embodiment, the implant is injected via needle subcutaneously in the posterior of the ear of the animal. The implanter used to inject the needle may be any of those commonly used in the art, with an implanter equipped with a hypodermic needle being particularly preferred.

The implant composition of the present invention can be used to deliver the active ingredient on an immediate and a sustained release basis to the following types of animals: cows, horses, sheep, swine, dogs, cats or any other suitable animal, including humans. In particularly preferred embodiments the implant containing differentially releasing MGA and/or TBA is injected into a heifer.

To use the implant of the present invention, the implant composition containing the immediate and sustained release vehicles is first prepared and then packaged for injectable use, typically as a magazine. Thereafter, the magazine is inserted into the implanter housing and the operator activates the implanter to puncture the skin of the animal. This is typically accomplished by a hypodermic needle. The implant composition thereafter traverses through the bore of the needle and into the puncture site. The operator thereafter withdraws the needle, leaving the implant device in the animal. Because of the physical or chemical nature of the immediate-release vehicle, the active is immediately released to the body and once distributed into the body is able to achieve an immediate and desired result. For example in a heifer, an immediate-release of substantial amount of MGA (e.g., in one pellet) can immediately inhibit pregnancy of the heifer. Because of the physical or chemical nature of the sustained release vehicle, the same active is distributed to the animal over a desired period of time (e.g., in five pellets). Using the above example, the sustained release of MGA can inhibit pregnancy for an extended period of time.

In the preferred embodiment where MGA (either alone or in combination with other actives) is contained in differential releasing pellets, the composition is capable of providing immediate and sustained release properties so that one injection will yield desired results in the animal first, immediately, and then for between about 60 to about 365 days with a more preferred range of from about 150 to about 200 days and a most preferred range of from about 180 to about 200 days.

By utilizing the implant composition and method as claimed herein, the following advantages are provided to the operator: dual effect by using the same biologically active material, modification of release rate providing for both immediate and sustained duration of effectiveness, potential reduction of residues that would occur if only one type of vehicle were used and treatment dosage only for the desired duration since a larger-than-optimal dose is not needed in order to achieve a rapid-onset of action, and possible carcass improvement in the case where the animal subject to treatment is a food animal.

The invention is further described in the following non-limiting examples.

### EXAMPLE 1

Two sets of biologically active pellets are formulated by conventional tableting technology, such as wet granulation with water as a granulation liquid or dry granulation, followed by screening, sizing and tablet compression.

| | Mg per | |
|---|---|---|
| Component | pellet | |
| Immediate-Release Pellets: | | |
| Melengestrol acetate Micronized | 24 | mg |
| Lactose Monohydrate NF Bolted | 5.0 | mg |
| Crosscaramellose Sodium NF Type A | 1.5 | mg |
| Pregelatinized Starch NF | 6.0 | mg |
| Colloidal Silicon Dioxide NF | 0.2 | mg |
| Magnesium Stearate NF Powder Food Grade | 1.0 | mg |
| Sustained-Release Pellets: | | |
| Melengestrol acetate Micronized | 24 | mg |
| Lactose Monohydrate NF Bolted | 8.235 | mg |
| Sorbitol NF Crystalline | 0.355 | mg |
| Sucrose NF Granular | 0.2755 | |
| Pregelatinized Starch NF | 2.0 | mg |
| Colloidal Silicon Dioxide NF | 0.2 | mg |
| Magnesium Stearate NF Powder Food Grade | 1.0 | mg |

### Release Characteristics of the Inventive Compositions

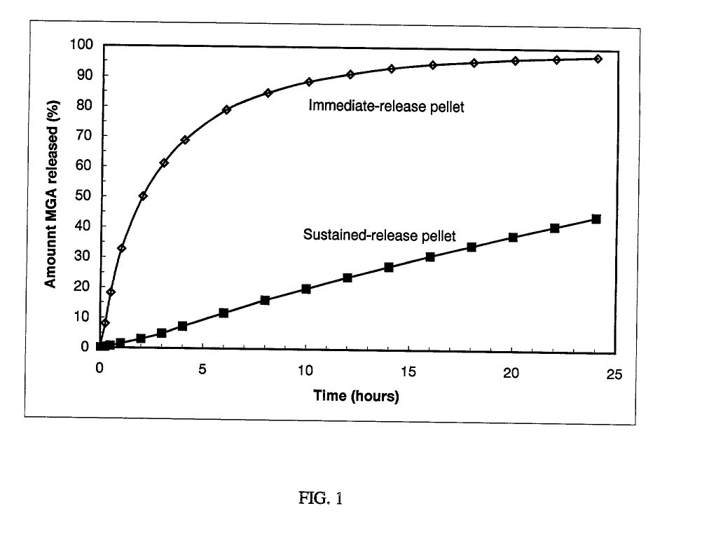

In-vitro release characteristics of the rapid-release and slow-release pellets of Example 1 are shown in FIG. 1 for dissolution testing carried out in a USP dissolution apparatus No. II (Paddle) at 37° C., in a dissolution medium composed of 0.3% SDS (sodium dodecyl sulfate), at 25 rpm. Referring to FIG. 1, the combining of the immediate-release and sustained-release pellets in different proportions in the same implant dose will allow for a wide range of in-vitro release profiles to be created, and thereby giving a range of in-vivo release rates. For the same total dose of active agent, an implant comprising of a larger number of rapid-releasing pellets, when compared to another comprising fewer of the rapid-releasing pellets, will provide a more rapid onset of action and also a shorter total duration of effect.

### Use of the Inventive Compositions

One or more of each of the immediate-release and sustained-release pellets of Example 1 are inserted into the magazine of an implanter device containing a hypodermic needle. For example, the implant may contain one immediate-release pellet and five sustained-release pellets. The operator activates the implanter to first puncture the skin, then deliver the implant composition through the needle and into the animal. In the case where the animal is a heifer, it is preferred that the puncture occurs at the posterior portion of the ear. The immediate-release pellet of the implant delivers the MGA in an amount of and rate sufficient to immediately inhibit pregnancy. The sustained-release pellets of the implant delivers the MGA in an amount of and rate sufficient to deliver to the heifer on a sustained release basis in order to exhibit growth increase, estrus suppression and inhibit pregnancy for an additional time period of from 150 to 200 days.

Various modifications of the present invention can be made without departing from the spirit or scope thereof and it should be understood that the invention is intended to be limited only as defined in the appended claims.

What is claimed is:

1. An implant composition comprising:
(a) a first component comprising a biologically active composition contained in a first delivery vehicle capable of immediately releasing said biologically active composition upon implantation in an animal body; and
(b) a second component comprising the same biologically active composition as in component (a) contained in a second delivery vehicle capable of releasing said biologically active composition on a sustained basis upon implantation in an animal body;
wherein said implant composition is implanted in an animal body by injection.

2. The implant composition of claim 1 wherein said first delivery vehicle is selected from the group consisting of encapsulants where the coating wall material is very thin, encapsulants where the coating wall material is highly soluble in body fluids, porous or freeze-dried solid compositions, solid tablets or pellets containing a disintegrating agent which causes the solid tablet or pellet to rapidly break down when in body fluids, solid tablets or pellets containing said biologically active material in fine or micronized particle sizes, an osmotic delivery system where the osmotic system is such that a substantial amount of the active is released upon implantation and mixtures thereof.

3. The implant composition of claim 1 wherein said second delivery vehicle is selected from the group consisting of encapsulated solutions or suspensions, biodegradable solid substances, conventional tablet/pellet ingredients, conventional tablet/pellet ingredients coated with a polymeric membrane to control release, conventional tablets or pellets containing said biologically active material having large particle sizes, matrix-tablets based on gel-forming excipients, matrix-type systems based on non-biodegradable polymers, membrane-type systems based on non-biodegradable polymers , matrix-type systems based on biodegradable polymers, matrix-type systems implant based on lipidic excipients, mass transfer systems based on osmotic pressure pumping through a hole in an impermeable coating and mixtures thereof.

4. The implant composition of claim 1 wherein the first delivery vehicle comprises solid tablets or pellets containing a disintegrating agent and wherein the second vehicle comprises solid tablets or pellets not containing a disintegrating agent.

5. The implant composition of claim 4, wherein said disintegrating agent is selected from the group consisting of sodium crosscaramellose, microcrystalline cellulose, sodium carboxymethyl-cellulose, alginic acid, starch, potassium polacrilin, colloidal silicon dioxide, crospovidone, guar gum, magnesium aluminum silicate, methyl cellulose, powdered cellulose, pregelatinized starch, sodium starch glycolate and sodium alginate and mixtures thereof.

6. The implant composition of claim 1 wherein said biologically active composition is selected from the group consisting of enzymes or other organic catalysts, ribozymes, organometalics, proteins and glycoproteins, peptides, poly(amino acids), antibodies, nucleic acids, steroids, antibiotics, antimycotics, anti-narcotics, cytostatics, cytotoxics, cytokines, carbohydrates, oleophobics, lipids, antihistamines, laxatives, vitamins, decongestants, gastrointestinal sedatives, anti-inflammatory substances, antimanics, anti-infectives, coronary vasodilators, peripheral vasodilators, cerebral vasodilators, psychotropics, stimulants, anti-diarrheal preparations, anti-anginal drugs, vasoconstrictors, anticoagulants, antithrombotic drugs, analgesics, antipyretics, hypnotics, sedatives, antiemetics, antinauseants, anticonvulsants, neuromuscular drugs, hyperglycemic and hypoglycemic agents, antivirals, antineoplastics antidepressants, anticholinergics, antiallergic agents, antidiabetic agents, antiarrythmics, antihormones, antihistamines, β-blockers, cardiac glycosides, contraceptives, contrast materials, radiopharmaceuticals, dopaminergic agents, lipid-regulating agents, uricoscurics, tranquilizers, thyroid and antithyroid preparations, diuretics, antispasmodics, uterine relaxants, mineral and nutritional additives, antiobesity drugs, microorganisms, viruses, releasing factors, growth factors, hormones, antihelmentics, steroids, and mixtures thereof.

7. The implant composition of claim 6 wherein said biologically active composition comprises a steroid, a hormone or mixtures thereof.

8. The implant composition of claim 7 wherein said biologically active composition comprises MGA, a combination of MGA and TBA or a combination of MGA, TBA and estradiol.

9. The implant composition of claim 8, wherein the MGA is contained in each delivery vehicle in an amount of from about 5 to about 200 mg per delivery vehicle.

10. The implant composition of claim 1 wherein either component (a) or component (b) or both further comprises one or more of the following materials: standard granulating aids, lubricants, diluents, binders and glidants, magnesium stearate, stearic acid, colloidal silicon dioxide, talc, titanium dioxide, magnesium, calcium and aluminum salts, lactose, cyclodextrins and derivatives thereof, starches, povidone, high molecular weight polyethylene glycols and derivatives thereof, bioerodible polymers and co-polymers, polystearates, carboxymethyl cellulose, cellulose, N,N-diethylamine acetate, polyvinyl alcohol, hydroxypropyl methyl cellulose, other biologically active or inactive substances or other pharmaceutically active or inactive substances.

11. An implant composition consisting essentially of:
(a) a first component comprising MGA contained in one or more pellets or tablets capable of immediately releasing said MGA upon implantation in an animal body, said pellet or tablet containing a disintegrating agent; and
(b) a second component comprising MGA contained in one or more pellets or tablets capable of releasing said biologically active composition on a sustained basis upon implantation in an animal body, said pellet or tablet not containing a disintegrating agent;
wherein said implant composition is implanted in an animal body by injection.

12. The implant of claim 11 consisting essentially of one to four pellets of type (a) and four to six pellets of type (b) which is administered by a single injection.

13. A method for delivering the same biologically active material to an animal body in both a rapid release and sustained release form comprising the steps of:
(1) providing an implant comprising:
(a) a first component comprising a biologically active composition contained in a first delivery vehicle capable of immediately releasing said biologically active composition upon implantation in an animal body; and
(b) a second component comprising the same biologically active composition as in component (a) contained in a second delivery vehicle capable of releasing said biologically active composition on a sustained basis upon implantation in an animal body; and
(2) injecting said implant into the animal body.

14. The method of claim 13 wherein the first delivery vehicle comprises solid tablets or pellets containing a disintegrating agent and wherein the second vehicle comprises solid tablets or pellets not containing a disintegrating agent.

15. The method of claim 14, wherein said disintegrating agent is selected from the group consisting of sodium crosscaramellose, microcrystalline cellulose, sodium carboxymethyl-cellulose, alginic acid, starch, potassium polacrilin, colloidal silicon dioxide, crospovidone, guar gum, magnesium aluminum silicate, methyl cellulose, powdered cellulose, pregelatinized starch, sodium starch glycolate and sodium alginate and mixtures thereof.

16. The method of claim 13 wherein said first delivery vehicle is selected from the group consisting of encapsulants where the coating wall material is very thin, encapsulants where the coating wall material is highly soluble in body fluids, porous solid compositions, solid tablets or pellets containing a disintegrating agent which causes the solid tablet or pellet to rapidly break down when in body fluids, solid tablets or pellets containing said biologically active material in fine or micronized particle sizes, an osmotic delivery system where the osmotic system is such that a substantial amount of the active is released upon implantation and mixtures thereof; and wherein said second delivery vehicle is selected from the group consisting of encapsulated solutions or suspensions, biodegradable solid substances, conventional tablet/pellet ingredients, conventional tablet/pellet ingredients coated with a polymeric membrane to control release, conventional tablets or pellets containing said biologically active material having large particle sizes, matrix-tablets based on gel-forming excipients, matrix-type systems based on non-biodegradable polymers, membrane-type systems based on non-biodegradable polymers, matrix-type systems based on biodegradable polymers, matrix-type systems based on lipidic excipients, mass transfer systems based on osmotic pressure pumping through a hole in an impermeable coating and mixtures thereof.

17. The method of claim 13, wherein said biologically active composition is selected from the group consisting of enzymes or other organic catalysts, ribozymes, organometalics, proteins and glycoproteins, peptides, poly(amino acids), antibodies, nucleic acids, steroids, antibiotics, antimycotics, anti-narcotics, cytostatics, cytotoxics, cytokines, carbohydrates, oleophobics, lipids, antihistamines, laxatives, vitamins, decongestants, gastrointestinal sedatives, anti-inflammatory substances, antimanics, anti-infectives, coronary vasodilators, peripheral vasodilators, cerebral vasodilators, psychotropics, stimulants, anti-diarrheal preparations, anti-anginal drugs, vasoconstrictors, anticoagulants, antithrombotic drugs, analgesics, antipyretics, hypnotics, sedatives, antiemetics, antinauseants, anticonvulsants, neuromuscular drugs, hyperglycemic and hypoglycemic agents, antivirals, antineoplastics antidepressants, anticholinergics, antiallergic agents, antidiabetic agents, antiarrythmics, antihormones, antihistamines, β-blockers, cardiac glycosides, contraceptives, contrast materials, radiopharmaceuticals, dopaminergic agents, lipid-regulating agents, uricoscurics, tranquilizers, thyroid and antithyroid preparations, diuretics, antispasmodics, uterine relaxants, mineral and nutritional additives, antiobesity drugs, microorganisms, viruses, releasing factors, growth factors, hormones, antihelmentics, steroids, and mixtures thereof.

18. The method of claim 17 wherein said biologically active composition comprises a steroid, a hormone or mixtures thereof.

19. The method of claim 18 wherein said biologically active composition comprises MGA, a combination of MGA and TBA or a combination of MGA, TBA and estradiol.

20. The method of claim 19, wherein the MGA is contained in each delivery vehicle in an amount of from about 5 to about 200 mg per delivery vehicle.

21. The method of claim 13, wherein said animal is selected from the group consisting of cows, horses, sheep, swine, dogs, cats and humans.

22. The method of claim 21, wherein said animal is a heifer.

23. The method of claim 13 wherein said implanting step is selected from the group consisting of subcutaneous, intramuscular, intraperitoneal, and intracranial injections.

24. The method of claim 23 wherein said animal is a heifer and said implanting step comprises subcutaneous injection in the posterior of the ear of said heifer.

25. The method of claim 13 wherein step (2) comprises a single injection.
